(19) United States
(12) Patent Application Publication
(10) Pub. No.: US 2022/0211672 A1
(43) Pub. Date: Jul. 7, 2022

(54) METHODS OF TREATING PRURITUS

(71) Applicant: CLEXIO BIOSCIENCES LTD., Jerusalem (IL)

(72) Inventors: Yael ROSEN, Givat Shaul (IL); David DANGOOR, Givat Shaul (IL); Richard FISHER, Givat Shaul (IL)

(21) Appl. No.: 17/607,632

(22) PCT Filed: May 1, 2020
(86) PCT No.: PCT/IB2020/054145

§ 371 Date: Oct. 29, 2021

Related U.S. Application Data
(60) Provisional application No. 62/841,373, filed on May 1, 2019.
(60) Provisional application No. 62/978,356, filed on Feb. 19, 2020.
(60) Provisional application No. 62/987,996, filed on Mar. 11, 2020.

(51) Int. Cl. A61K 31/4174; A61K 31/4168; A61K 31/498; A61K 31/155; A61K 31/54; A61P 17/04; A61K 9/00; A61K 31/335; A61K 31/436; A61K 31/167; A61K 31/135; A61K 31/045; A61K 31/496; A61K 31/69; A61K 31/454; A61K 31/4164; A61K 31/195; A61K 31/485

(52) U.S. Cl. CPC A61K 31/4174; A61K 31/485; A61K 31/498; A61K 31/155; A61K 31/54; A61P 17/04; A61K 9/0014; A61K 31/335; A61K 31/436; A61K 31/167; A61K 31/135; A61K 31/045; A61K 31/496; A61K 31/69; A61K 31/454; A61K 31/4164; A61K 31/195; A61K 31/4168

(57) ABSTRACT

The present disclosure related to methods of treating pruritus in a subject by topically administering an alpha-2 adrenoreceptor agonist to a subject.

### TECHNICAL FIELD

The present disclosure related to methods of treating pruritus in a subject by topically administering an alpha-2 adrenoreceptor agonist to a subject.

### BACKGROUND

Pruritus is an unpleasant sensation that provokes the desire to scratch. The condition is extremely common with estimates that at any given time between 8 and 16% of adults are suffering from it, resulting in significant reductions in quality of life for sufferers. To date, despite numerous attempts at clinical studies, no drug has been approved to treat the condition.

Apraclonidine is a synthetic alpha-2 adrenoreceptor agonist with analgesic properties. It is presently sold by prescription under the trade name IOPIDINE® (Alcon, Fort Worth, Tex.) as a sterile isotonic solution for topical ophthalmic application to control or prevent post-surgical elevations in intraocular pressure (IOP).

Brimonidine is a synthetic alpha-2 adrenoreceptor agonist with sedative and analgesic properties. It is presently sold by prescription under the trade names ALPHAGAN® (Allergan, Irvine, Calif.) and QOLIANA® (Alcon, Fort Worth, Tex.), as ophthalmic solutions for preventing elevation in IOP, LUMIFY® (Bausch and Lomb, Bridgewater, N.J.) as redness reliever eye drops and MIRVASO® (Galderma, Fort Worth, Tex.) as a topical gel for treating the facial erythema of rosacea.

Clonidine is a synthetic alpha-2 adrenoreceptor agonist with sedative and analgesic properties. It is presently sold by prescription under the trade name CATAPRES® (Boehringer Ingelheim, Ridgefield, Conn.) as a centrally acting alpha-agonist hypotensive agent and as DURACLON® (Mylan, Lake Forest, Ill.) as a centrally-acting analgesic solution for use in continuous epidural infusion devices.

Detomidine is a synthetic alpha-2 adrenoreceptor agonist with sedative and analgesic properties. It is presently sold by prescription under the trade name DORMOSEDAN® (Zoetis Services LLC, Parsippany, N.J.) as a sedative and anesthetic premedication for horses and other large animals. It is commonly combined with butorphanol in order to increase the degree of analgesia and depth of sedation, and may also be used with ketamine for intravenous anesthesia of short duration. The route of administration of DORMOSEDAN® injection is typically intramuscular or intravenous, but the drug is also available as a gel (DORMOSEDAN GEL®) that may be administered by the sublingual route. More recently, detomidine has been shown to be effective as a topical analgesic agent.

Medetomidine and its single enantiomer derivative, Dexmedetomidine, are synthetic alpha-2 adrenoreceptor agonists with sedative and analgesic properties. Medetomidine is presently sold by prescription under the trade name DOMITOR® (Zoetis Services LLC, Parsippany, N.J.) as an intramuscular or intravenous injection for sedation or analgesia for cats and dogs. Dexmedetomidine is presently sold by prescription under the trade name PRECEDEX® (Hospira, Lake Forest, Ill.) as an intravenous injection for sedation of patients.

Guanabenz is a synthetic alpha-2 adrenoreceptor agonist with sedative and analgesic properties. It is currently unavailable in the US, but was previously sold by prescription under the trade name WYTENSIN® (Wyeth-Ayerst) as an oral antihypertensive agent.

Lofexidine is a synthetic alpha-2 adrenoreceptor agonist with sedative and analgesic properties. It is presently sold by prescription under the trade name LUCEMYRA® (US WorldMeds, Louisville, Ky.) as a tablet for the mitigation of opioid withdrawal symptoms.

Romifidine is a synthetic alpha-2 adrenoreceptor agonist with sedative and analgesic properties. It is presently sold by prescription under the trade name SEDIVET® (Boehringer Ingelheim, St. Joseph, Mo.) as an injectable sedative and analgesic for horses.

Xylazine is a synthetic alpha-2 adrenoreceptor agonist with sedative and analgesic properties. It is presently sold by prescription under the trade name ROMPUN® (Bayer, Shawnee Mission, Kans.) as an injectable sedative and analgesic for horses and Cervidae.

### SUMMARY OF THE INVENTION

The present invention relates to the topical treatment of pruritus by an alpha-2 adrenoreceptor agonist.

### BRIEF DESCRIPTION OF THE DRAWINGS

FIG. 1: A graphical representation of scratching events over time.

FIG. 2: Photographs of immunostaining of pig skin biopsies at ×200 (a) and ×400 (b, c) magnification.

### DETAILED DESCRIPTION

The present inventions may be understood more readily by reference to the following detailed description taken in connection with any accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions. The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a particle” is a reference to one or more of such particles and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours etc. . . . up to 5 hours.

“Subject,” as used herein, includes humans and animals. The terms “human,” “patient,” and “subject” are used interchangeably herein.

The present invention relates to methods of treating pruritus in a subject comprising topically administering to the subject's skin a pharmaceutical composition comprising an alpha-2 adrenoreceptor agonist. According to the invention, the amount of an alpha-2 adrenoreceptor agonist topically administered to the subject is sufficient to treat pruritus. Pruritus can be demonstrated to have been treated by reductions in VAS, NRS, Quality of Life and/or pruritus scores or by other methods known in the art.

In one embodiment, the topically administered alpha-2 adrenoreceptor agonist to treat pruritus is apraclonidine, brimonidine, clonidine, detomidine, dexmedetomidine, guanabenz, lofexidine, medetomidine, romifidine or xylazine. According to the invention, the amount of an alpha-2 adrenoreceptor agonist, as selected from apraclonidine, brimonidine, clonidine, detomidine, dexmedetomidine, guanabenz, lofexidine, medetomidine, romifidine or xylazine, topically administered to the subject is sufficient to treat pruritus.

Apraclonidine may be topically administered as the free base form or as a salt. Unless specified otherwise, reference to “apraclonidine” in the present disclosure can refer to apraclonidine in a free base form, or to a salt of apraclonidine. Those of ordinary skill in the art can readily identify exemplary pharmaceutically acceptable salt forms of apraclonidine. Suitable pharmaceutically acceptable salts of apraclonidine include apraclonidine bitartrate, apraclonidine bitartrate hydrate, apraclonidine hydrochloride, apraclonidine p-toluenesulfonate, apraclonidine phosphate, apraclonidine thiosemicarbazone, apraclonidine sulfate, apraclonidine trifluoroacetate, apraclonidine hemipentahydrate, apraclonidine bitartrate hemipentahydrate, apraclonidine pentafluoropropionate, apraclonidine p-nitrophenylhydrazone, apraclonidine o-methyloxime, apraclonidine semicarbazone, apraclonidine hydrobromide, apraclonidine mucate, apraclonidine oleate, apraclonidine phosphate dibasic, apraclonidine phosphate monobasic, apraclonidine inorganic salt, apraclonidine organic salt, apraclonidine acetate trihydrate, apraclonidine bis(heptafluorobutyrate), apraclonidine bis(methylcarbamate), apraclonidine bis(pentafluoropropionate), apraclonidine bis(pyridine carboxylate), apraclonidine bis(trifluoroacetate), apraclonidine chlorhydrate, and apraclonidine sulfate pentahydrate. In certain embodiments of the presently disclosed dosage forms, the apraclonidine is present as the hydrochloride salt.

Brimonidine may be topically administered as the free base form or as a salt. Unless specified otherwise, reference to “brimonidine” in the present disclosure can refer to brimonidine in a free base form, or to a salt of brimonidine. Those of ordinary skill in the art can readily identify exemplary pharmaceutically acceptable salt forms of brimonidine. Suitable pharmaceutically acceptable salts of brimonidine include brimonidine tartrate, brimonidine tartrate hydrate, brimonidine hydrochloride, brimonidine p-toluenesulfonate, brimonidine phosphate, brimonidine thiosemicarbazone, brimonidine sulfate, brimonidine trifluoroacetate, brimonidine hemipentahydrate, brimonidine tartrate hemipentahydrate, brimonidine pentafluoropropionate, brimonidine p-nitrophenylhydrazone, brimonidine o-methyloxime, brimonidine semicarbazone, brimonidine hydrobromide, brimonidine mucate, brimonidine oleate, brimonidine phosphate dibasic, brimonidine phosphate monobasic, brimonidine inorganic salt, brimonidine organic salt, brimonidine acetate trihydrate, brimonidine bis(heptafluorobutyrate), brimonidine bis(methylcarbamate), brimonidine bis(pentafluoropropionate), brimonidine bis(pyridine carboxylate), brimonidine bis(trifluoroacetate), brimonidine chlorhydrate, and brimonidine sulfate pentahydrate. In certain embodiments of the presently disclosed dosage forms, the brimonidine is present as the tartrate salt.

Clonidine may be topically administered as the free base form or as a salt. Unless specified otherwise, reference to “clonidine” in the present disclosure can refer to clonidine in a free base form, or to a salt of clonidine. Those of ordinary skill in the art can readily identify exemplary pharmaceutically acceptable salt forms of clonidine. Suitable pharmaceutically acceptable salts of clonidine include clonidine bitartrate, clonidine bitartrate hydrate, clonidine hydrochloride, clonidine p-toluenesulfonate, clonidine phosphate, clonidine thiosemicarbazone, clonidine sulfate, clonidine trifluoroacetate, clonidine hemipentahydrate, clonidine bitartrate hemipentahydrate, clonidine pentafluoropropionate, clonidine p-nitrophenylhydrazone, clonidine o-methyloxime, clonidine semicarbazone, clonidine hydrobromide, clonidine mucate, clonidine oleate, clonidine phosphate dibasic, clonidine phosphate monobasic, clonidine inorganic salt, clonidine organic salt, clonidine acetate trihydrate, clonidine bis(heptafluorobutyrate), clonidine bis(methylcarbamate), clonidine bis(pentafluoropropionate), clonidine bis(pyridine carboxylate), clonidine bis(trifluoroacetate), clonidine chlorhydrate, and clonidine sulfate pentahydrate. In certain embodiments of the presently disclosed dosage forms, the clonidine is present as the hydrochloride salt.

Detomidine may be topically administered as the free base form or as a salt. Unless specified otherwise, reference to “detomidine” in the present disclosure can refer to detomidine in a free base form, or to a salt of detomidine. Those of ordinary skill in the art can readily identify exemplary pharmaceutically acceptable salt forms of detomidine. Suitable pharmaceutically acceptable salts of detomidine include detomidine bitartrate, detomidine bitartrate hydrate, detomidine hydrochloride, detomidine p-toluenesulfonate, detomidine phosphate, detomidine thiosemicarbazone, detomidine sulfate, detomidine trifluoroacetate, detomidine hemipentahydrate, detomidine bitartrate hemipentahydrate, detomidine pentafluoropropionate, detomidine p-nitrophenylhydrazone, detomidine o-methyloxime, detomidine semicarbazone, detomidine hydrobromide, detomidine mucate, detomidine oleate, detomidine phosphate dibasic, detomidine phosphate monobasic, detomidine inorganic salt, detomidine organic salt, detomidine acetate trihydrate, detomidine bis(heptafluorobutyrate), detomidine bis(methylcarbamate), detomidine bis(pentafluoropropionate), detomidine bis(pyridine carboxylate), detomidine bis(trifluoroacetate), detomidine chlorhydrate, and detomidine sulfate pentahydrate. In certain embodiments of the presently disclosed dosage forms, the detomidine is present as the hydrochloride salt. In certain embodiments, the detomidine is anhydrous detomidine hydrochloride. In other embodiments, the detomidine is detomidine hydrochloride monohydrate.

Medetomidine may be topically administered as the free base form or as a salt. Unless specified otherwise, reference to “medetomidine” in the present disclosure can refer to medetomidine in a free base form, or to a salt of medetomidine. Those of ordinary skill in the art can readily identify exemplary pharmaceutically acceptable salt forms of medetomidine. Suitable pharmaceutically acceptable salts of medetomidine include medetomidine bitartrate, medetomidine bitartrate hydrate, medetomidine hydrochloride, medetomidine p-toluenesulfonate, medetomidine phosphate, medetomidine thiosemicarbazone, medetomidine sulfate, medetomidine trifluoroacetate, medetomidine hemipentahydrate, medetomidine bitartrate hemipentahydrate, medetomidine pentafluoropropionate, medetomidine p-nitrophenylhydrazone, medetomidine o-methyloxime, medetomidine semicarbazone, medetomidine hydrobromide, medetomidine mucate, medetomidine oleate, medetomidine phosphate dibasic, medetomidine phosphate monobasic, medetomidine inorganic salt, medetomidine organic salt, medetomidine acetate trihydrate, medetomidine bis(heptafluorobutyrate), medetomidine bis(methylcarbamate), medetomidine bis(pentafluoropropionate), medetomidine bis(pyridine carboxylate), medetomidine bis(trifluoroacetate), medetomidine chlorhydrate, and medetomidine sulfate pentahydrate. In certain embodiments of the presently disclosed dosage forms, the medetomidine is present as the hydrochloride salt.

Dexmedetomidine may be topically administered as the free base form or as a salt. Unless specified otherwise, reference to “dexmedetomidine” in the present disclosure can refer to dexmedetomidine in a free base form, or to a salt of dexmedetomidine. Those of ordinary skill in the art can readily identify exemplary pharmaceutically acceptable salt forms of dexmedetomidine. Suitable pharmaceutically acceptable salts of dexmedetomidine include dexmedetomidine bitartrate, dexmedetomidine bitartrate hydrate, dexmedetomidine hydrochloride, dexmedetomidine p-toluenesulfonate, dexmedetomidine phosphate, dexmedetomidine thiosemicarbazone, dexmedetomidine sulfate, dexmedetomidine trifluoroacetate, dexmedetomidine hemipentahydrate, dexmedetomidine bitartrate hemipentahydrate, dexmedetomidine pentafluoropropionate, dexmedetomidine p-nitrophenylhydrazone, dexmedetomidine o-methyloxime, dexmedetomidine semicarbazone, dexmedetomidine hydrobromide, dexmedetomidine mucate, dexmedetomidine oleate, dexmedetomidine phosphate dibasic, dexmedetomidine phosphate monobasic, dexmedetomidine inorganic salt, dexmedetomidine organic salt, dexmedetomidine acetate trihydrate, dexmedetomidine bis(heptafluorobutyrate), dexmedetomidine bis(methylcarbamate), dexmedetomidine bis(pentafluoropropionate), dexmedetomidine bis(pyridine carboxylate), dexmedetomidine bis(trifluoroacetate), dexmedetomidine chlorhydrate, and dexmedetomidine sulfate pentahydrate. In certain embodiments of the presently disclosed dosage forms, the dexmedetomidine is present as the hydrochloride salt.

Guanabenz may be topically administered as the free base form or as a salt. Unless specified otherwise, reference to “guanabenz” in the present disclosure can refer to guanabenz in a free base form, or to a salt of guanabenz. Those of ordinary skill in the art can readily identify exemplary pharmaceutically acceptable salt forms of guanabenz. Suitable pharmaceutically acceptable salts of guanabenz include guanabenz tartrate, guanabenz tartrate hydrate, guanabenz acetate, guanabenz hydrochloride, guanabenz p-toluenesulfonate, guanabenz phosphate, guanabenz thiosemicarbazone, guanabenz sulfate, guanabenz trifluoroacetate, guanabenz hemipentahydrate, guanabenz bitartrate hemipentahydrate, guanabenz pentafluoropropionate, guanabenz p-nitrophenylhydrazone, guanabenz o-methyloxime, guanabenz semicarbazone, guanabenz hydrobromide, guanabenz mucate, guanabenz oleate, guanabenz phosphate dibasic, guanabenz phosphate monobasic, guanabenz inorganic salt, guanabenz organic salt, guanabenz acetate trihydrate, guanabenz bis(heptafluorobutyrate), guanabenz bis(methylcarbamate), guanabenz bis(pentafluoropropionate), guanabenz bis(pyridine carboxylate), guanabenz bis(trifluoroacetate), guanabenz chlorhydrate, and guanabenz sulfate pentahydrate. In certain embodiments of the presently disclosed dosage forms, the guanabenz is present as the acetate salt.

Lofexidine may be topically administered as the free base form or as a salt and in either racemic or enantiomeric form. Unless specified otherwise, reference to “lofexidine” in the present disclosure can refer to lofexidine in a free base form, or to a salt of lofexidine. Those of ordinary skill in the art can readily identify exemplary pharmaceutically acceptable salt forms of lofexidine. Suitable pharmaceutically acceptable salts of lofexidine include lofexidine tartrate, lofexidine tartrate hydrate, lofexidine acetate, lofexidine hydrochloride, lofexidine p-toluenesulfonate, lofexidine phosphate, lofexidine thiosemicarbazone, lofexidine sulfate, lofexidine trifluoroacetate, lofexidine hemipentahydrate, lofexidine bitartrate hemipentahydrate, lofexidine pentafluoropropionate, lofexidine p-nitrophenylhydrazone, lofexidine o-methyloxime, lofexidine semicarbazone, lofexidine hydrobromide, lofexidine mucate, lofexidine oleate, lofexidine phosphate dibasic, lofexidine phosphate monobasic, lofexidine inorganic salt, lofexidine organic salt, lofexidine acetate trihydrate, lofexidine bis(heptafluorobutyrate), lofexidine bis(methylcarbamate), lofexidine bis(pentafluoropropionate), lofexidine bis(pyridine carboxylate), lofexidine bis(trifluoroacetate), lofexidine chlorhydrate, and lofexidine sulfate pentahydrate. In certain embodiments of the presently disclosed dosage forms, the lofexidine is present as the hydrochloride salt.

Romifidine may be topically administered as the free base form or as a salt. Unless specified otherwise, reference to “romifidine” in the present disclosure can refer to romifidine in a free base form, or to a salt of romifidine. Those of ordinary skill in the art can readily identify exemplary pharmaceutically acceptable salt forms of romifidine. Suitable pharmaceutically acceptable salts of romifidine include romifidine bitartrate, romifidine bitartrate hydrate, romifidine hydrochloride, romifidine p-toluenesulfonate, romifidine phosphate, romifidine thiosemicarbazone, romifidine sulfate, romifidine trifluoroacetate, romifidine hemipentahydrate, romifidine bitartrate hemipentahydrate, romifidine pentafluoropropionate, romifidine p-nitrophenylhydrazone, romifidine o-methyloxime, romifidine semicarbazone, romifidine hydrobromide, romifidine mucate, romifidine oleate, romifidine phosphate dibasic, romifidine phosphate monobasic, romifidine inorganic salt, romifidine organic salt, romifidine acetate trihydrate, romifidine bis(heptafluorobutyrate), romifidine bis(methylcarbamate), romifidine bis(pentafluoropropionate), romifidine bis(pyridine carboxylate), romifidine bis(trifluoroacetate), romifidine chlorhydrate, and romifidine sulfate pentahydrate. In certain embodiments of the presently disclosed dosage forms, the romifidine is present as the hydrochloride salt.

Xylazine may be topically administered as the free base form or as a salt. Unless specified otherwise, reference to “xylazine” in the present disclosure can refer to xylazine in a free base form, or to a salt of xylazine. Those of ordinary skill in the art can readily identify exemplary pharmaceutically acceptable salt forms of xylazine. Suitable pharmaceutically acceptable salts of xylazine include xylazine bitartrate, xylazine bitartrate hydrate, xylazine hydrochloride, xylazine p-toluenesulfonate, xylazine phosphate, xylazine thiosemicarbazone, xylazine sulfate, xylazine trifluoroacetate, xylazine hemipentahydrate, xylazine bitartrate hemipentahydrate, xylazine pentafluoropropionate, xylazine p-nitrophenylhydrazone, xylazine o-methyloxime, xylazine semicarbazone, xylazine hydrobromide, xylazine mucate, xylazine oleate, xylazine phosphate dibasic, xylazine phosphate monobasic, xylazine inorganic salt, xylazine organic salt, xylazine acetate trihydrate, xylazine bis(heptafluorobutyrate), xylazine bis(methylcarbamate), xylazine bis(pentafluoropropionate), xylazine bis(pyridine carboxylate), xylazine bis(trifluoroacetate), xylazine chlorhydrate, and xylazine sulfate pentahydrate. In certain embodiments of the presently disclosed dosage forms, the xylazine is present as the hydrochloride salt.

In some embodiments, the pruritus is acute. Acute pruritus is the defined as the manifestation of the condition for up to six consecutive weeks. In other embodiments, the pruritus is chronic. Chronic pruritus is defined as the manifestation of the condition for more than six consecutive weeks.

The present invention relates to methods of treating pruritus in a subject comprising topically administering to the subject's skin a pharmaceutical composition comprising an alpha-2 adrenoreceptor agonist. In one embodiment, the pruritus is acute pruritus. In another embodiment, the pruritus is chronic pruritus.

In certain embodiments, the present invention relates to methods of treating pruritus in a subject comprising topically administering to the subject's skin a pharmaceutical composition comprising an alpha-2 adrenoreceptor agonist wherein the alpha-2 adrenoreceptor agonist is apraclonidine, brimonidine, clonidine, detomidine, dexmedetomidine, guanabenz, lofexidine, medetomidine, romifidine or xylazine. In one embodiment, the pruritus treated by an alpha-2 adrenoreceptor agonist apraclonidine, brimonidine, clonidine, detomidine, dexmedetomidine, guanabenz, lofexidine, medetomidine, romifidine or xylazine, is acute pruritus. In another embodiment, the pruritus treated by an alpha-2 adrenoreceptor agonist that is apraclonidine, brimonidine, clonidine, detomidine, dexmedetomidine, guanabenz, lofexidine, medetomidine, romifidine or xylazine, is chronic pruritus.

In certain embodiments, the origin of the pruritus is unknown. In other embodiments, the pruritus comprises the symptom of a dermatological, neurological, psychogenic or systemic condition, or is of mixed origin.

The present invention relates to methods of treating pruritus in a subject comprising topically administering to the subject's skin a pharmaceutical composition comprising an alpha-2 adrenoreceptor agonist and wherein the pruritus is of unknown origin. In another embodiment, the present invention relates to methods of treating pruritus in a subject comprising topically administering to the subject's skin a pharmaceutical composition comprising an alpha-2 adrenoreceptor agonist and wherein the pruritus comprises the symptom of a dermatological, neurological, psychogenic or systemic condition, or is of mixed origin.

In certain embodiments, the present invention relates to methods of treating pruritus in a subject comprising topically administering to the subject's skin a pharmaceutical composition comprising an alpha-2 adrenoreceptor agonist that is apraclonidine, brimonidine, clonidine, detomidine, dexmedetomidine, guanabenz, lofexidine, medetomidine, romifidine or xylazine and wherein the pruritus is of unknown origin. In certain embodiments, the present invention relates to methods of treating pruritus in a subject comprising topically administering to the subject's skin a pharmaceutical composition comprising an alpha-2 adrenoreceptor agonist that is apraclonidine, brimonidine, clonidine, detomidine, dexmedetomidine, guanabenz, lofexidine, medetomidine, romifidine or xylazine and wherein the pruritus comprises the symptom of a dermatological, neurological, psychogenic or systemic condition, or is of mixed origin.

In certain embodiments, the present invention relates to methods of treating pruritus in a subject comprising topically administering to the subject's skin a pharmaceutical composition comprising an alpha-2 adrenoreceptor agonist that is apraclonidine, brimonidine, clonidine, detomidine, dexmedetomidine, guanabenz, lofexidine, medetomidine, romifidine or xylazine and wherein the pruritus comprises the symptom of dermatological origin.

In certain embodiments, the present invention relates to methods of treating pruritus in a subject comprising topically administering to the subject's skin a pharmaceutical composition comprising an alpha-2 adrenoreceptor agonist that is apraclonidine, brimonidine, clonidine, detomidine, dexmedetomidine, guanabenz, lofexidine, medetomidine, romifidine or xylazine and wherein the pruritus comprises the symptom of neurological origin.

In certain embodiments, the present invention relates to methods of treating pruritus in a subject comprising topically administering to the subject's skin a pharmaceutical composition comprising an alpha-2 adrenoreceptor agonist that is apraclonidine, brimonidine, clonidine, detomidine, dexmedetomidine, guanabenz, lofexidine, medetomidine, romifidine or xylazine and wherein the pruritus comprises the symptom of psychogenic origin.

In certain embodiments, the present invention relates to methods of treating pruritus in a subject comprising topically administering to the subject's skin a pharmaceutical composition comprising an alpha-2 adrenoreceptor agonist that is apraclonidine, brimonidine, clonidine, detomidine, dexmedetomidine, guanabenz, lofexidine, medetomidine, romifidine or xylazine and wherein the pruritus comprises the symptom of a systemic condition.

In certain embodiments, the present invention relates to methods of treating pruritus in a subject comprising topically administering to the subject's skin a pharmaceutical composition comprising an alpha-2 adrenoreceptor agonist that is apraclonidine, brimonidine, clonidine, detomidine, dexmedetomidine, guanabenz, lofexidine, medetomidine, romifidine or xylazine and wherein the pruritus comprises the symptom of mixed origin.

Examples of pruritic dermatological conditions include atopic dermatitis, contact dermatitis, allergic dermatitis, seborrheic dermatitis, statis dermatitis, pityriasis rubra pilaris, pityriasis rosea, acne, dermatitis herpetiformis, pemphigus vulgaris, bullous pemphigoid, lichen planus, prurigo nodularis, lichen simplex chronicus, lichen amyloidosis, urticaria, mastocytosis, polymorphous light eruption, actinic prurigo, chronic prurigo, actinic dermatitis, polymorphic eruption of pregnancy, eosinophilic folliculitis, dermatomyositis, prurigo pigmentosa, lichen sclerosus, palmoplantar pustulosis, pompholyx, idiopathic xerosis, scarring, burns, burn scars, keloid scars, hypertrophic scars, reactive drug eruptions and pruritus of an infestive or infective origin.

Examples of pruritic infestive conditions include scabies, pediculosis and arthropod bites.

Examples of pruritic infective conditions include fungal, parasitic, viral and bacterial conditions.

Examples of pruritic neurological conditions include notalgia paresthetica, brachioradial pruritus, postherpetic neuralgia, stroke, small fiber neuropathy, trigeminal trophic syndrome, Creutzfeldt-Jakob disease, chemotherapy-induced neuropathy, HIV-related neuropathy and multiple sclerosis.

Examples of pruritic psychogenic conditions include depression, anxiety, psychogenic excoriation, anorexia nervosa and delusional parasitosis.

Examples of pruritic systemic conditions include chronic renal failure, uremic pruritus, liver disease, primary biliary cholangitis, primary biliary cirrhosis, cholestatic jaundice, hepatitis C, cholestasis of pregnancy, polycythemia vera, iron deficiency anemia, Hodgkin lymphoma, non-Hodgkin lymphoma, multiple myeloma, hematologic or lymphoproliferative disorders, primary cutaneous lymphoma, mycosis fungoides, cutaneous T cell lymphoma, malignancy, plasma cell dyscrasias, gastric carcinoid tumors, hyperthyroidism, hypothyroidism, hyperparathyroidism, haemochromatosis, celiac disease, systemic lupus erythematosus, systemic sclerosis, diabetes, carcinoid syndrome, dermatomyositis, scleroderma, Sjögren's syndrome, linear immunoglobulin A (IgA) disease, graft-versus-host disease, Darier disease, Hailey-Hailey disease, porphyria and amyloidosis.

Pruritus is understood to occur when pruritogens activate receptors on small itch-selective unmyelinated C-fibers. Two subtypes of itch-sensitive neurons are found in the dermal tissues, histaminergic and non-histaminergic neurons, each with different tracts and different patterns of brain activation.

Histaminergic neurons are primarily involved in acute pruritus. Histamine is released by mast cells and other immune cells and keratinocytes. H1 and H4 receptors on histaminergic nerves bind histamine and activate TRPV1 through the phospholipase system. The excited histaminergic neurons also release neuropeptides such as calcitonin gene-related protein and substance P, which can cause inflammatory effects such as local vasodilation, plasma extravasation, and mast cell degranulation.

Non-histaminergic neurons can be excited by endogenous/exogeneous pruritogens other than histamine and express various receptors involved in pruritus. These receptors activate either TRPV1 or TRPA1 through the phospholipase or kinase system.

As shown in U.S. Pat. No. 8,026,266, US20150098982, WO2009158477, WO2011085162, WO2018129313, each of which are incorporated herein in its entirety, alpha-2 adrenoreceptor agonists have been demonstrated to be effective as topical agents for the treatment of pain.

Without wanting to be bound to any particular theory, it is believed that when administered topically, an alpha-2 adrenoreceptor agonist can inhibit peripheral pruritus signal transduction in a similar manner to its inhibition of peripheral pain signal transduction.

In certain embodiments, the alpha-2 adrenoreceptor agonist is administered topically in pharmaceutical compositions comprising about 0.01 to about 5 wt % of an alpha-2 adrenoreceptor agonist. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of an alpha-2 adrenoreceptor agonist. In certain embodiments, the pharmaceutical compositions comprise 0.033, 0.1, 0.33 or 1 wt % of an alpha-2 adrenoreceptor agonist. In a preferred embodiment, the pharmaceutical composition comprises 0.1 wt % of an alpha-2 adrenoreceptor agonist. In another preferred embodiment, the pharmaceutical composition comprises 0.33 wt % of an alpha-2 adrenoreceptor agonist. In another preferred embodiment, the pharmaceutical composition comprises 1 wt % of an alpha-2 adrenoreceptor agonist.

In certain embodiments, apraclonidine is administered topically in pharmaceutical compositions comprising about 0.01 to about 5 wt % of apraclonidine. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of apraclonidine. In certain embodiments, the pharmaceutical compositions comprise 0.033, 0.1, 0.33 or 1 wt % of apraclonidine. In a preferred embodiment, the pharmaceutical composition comprises 0.1 wt % of apraclonidine. In another preferred embodiment, the pharmaceutical composition comprises 0.33 wt % of apraclonidine. In another preferred embodiment, the pharmaceutical composition comprises 1 wt % of apraclonidine.

In certain embodiments, brimonidine is administered topically in pharmaceutical compositions comprising about 0.01 to about 5 wt % of brimonidine. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of brimonidine. In certain embodiments, the pharmaceutical compositions comprise 0.033, 0.1, 0.33 or 1 wt % of brimonidine. In a preferred embodiment, the pharmaceutical composition comprises 0.1 wt % of brimonidine. In another preferred embodiment, the pharmaceutical composition comprises 0.33 wt % of brimonidine. In another preferred embodiment, the pharmaceutical composition comprises 1 wt % of brimonidine.

In certain embodiments, clonidine is administered topically in pharmaceutical compositions comprising about 0.01 to about 5 wt % of clonidine. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of clonidine. In certain embodiments, the pharmaceutical compositions comprise 0.033, 0.1, 0.33 or 1 wt % of clonidine. In a preferred embodiment, the pharmaceutical composition comprises 0.1 wt % of clonidine. In another preferred embodiment, the pharmaceutical composition comprises 0.33 wt % of clonidine. In another preferred embodiment, the pharmaceutical composition comprises 1 wt % of clonidine.

In certain embodiments, detomidine is administered topically in pharmaceutical compositions comprising about 0.01 to about 5 wt % of detomidine. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of detomidine. In certain embodiments, the pharmaceutical compositions comprise 0.033, 0.1, 0.33 or 1 wt % of detomidine. In a preferred embodiment, the pharmaceutical composition comprises 0.1 wt % of detomidine. In another preferred embodiment, the pharmaceutical composition comprises 0.33 wt % of detomidine. In another preferred embodiment, the pharmaceutical composition comprises 1 wt % of detomidine.

In certain embodiments, medetomidine is administered topically in pharmaceutical compositions comprising about 0.01 to about 5 wt % of medetomidine. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of medetomidine. In certain embodiments, the pharmaceutical compositions comprise 0.033, 0.1, 0.33 or 1 wt % of medetomidine. In a preferred embodiment, the pharmaceutical composition comprises 0.1 wt % of medetomidine. In another preferred embodiment, the pharmaceutical composition comprises 0.33 wt % of medetomidine. In another preferred embodiment, the pharmaceutical composition comprises 1 wt % of medetomidine.

In certain embodiments, dexmedetomidine is administered topically in pharmaceutical compositions comprising about 0.01 to about 5 wt % of dexmedetomidine. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of dexmedetomidine. In certain embodiments, the pharmaceutical compositions comprise 0.033, 0.1, 0.33 or 1 wt % of dexmedetomidine. In a preferred embodiment, the pharmaceutical composition comprises 0.1 wt % of dexmedetomidine. In another preferred embodiment, the pharmaceutical composition comprises 0.33 wt % of dexmedetomidine. In another preferred embodiment, the pharmaceutical composition comprises 1 wt % of dexmedetomidine.

In certain embodiments, guanabenz is administered topically in pharmaceutical compositions comprising about 0.01 to about 5 wt % of guanabenz. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of guanabenz. In certain embodiments, the pharmaceutical compositions comprise 0.033, 0.1, 0.33 or 1 wt % of guanabenz. In a preferred embodiment, the pharmaceutical composition comprises 0.1 wt % of guanabenz. In another preferred embodiment, the pharmaceutical composition comprises 0.33 wt % of guanabenz. In another preferred embodiment, the pharmaceutical composition comprises 1 wt % of guanabenz.

In certain embodiments, lofexidine is administered topically in pharmaceutical compositions comprising about 0.01 to about 5 wt % of lofexidine. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of lofexidine. In certain embodiments, the pharmaceutical compositions comprise 0.033, 0.1, 0.33 or 1 wt % of lofexidine. In a preferred embodiment, the pharmaceutical composition comprises 0.1 wt % of lofexidine. In another preferred embodiment, the pharmaceutical composition comprises 0.33 wt % of lofexidine. In another preferred embodiment, the pharmaceutical composition comprises 1 wt % of lofexidine. In yet another preferred embodiment, the pharmaceutical composition comprises 2 wt % of lofexidine.

In certain embodiments, romifidine is administered topically in pharmaceutical compositions comprising about 0.01 to about 5 wt % of romifidine. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of romifidine. In certain embodiments, the pharmaceutical compositions comprise 0.033, 0.1, 0.33 or 1 wt % of romifidine. In a preferred embodiment, the pharmaceutical composition comprises 0.1 wt % of romifidine. In another preferred embodiment, the pharmaceutical composition comprises 0.33 wt % of romifidine. In another preferred embodiment, the pharmaceutical composition comprises 1 wt % of romifidine.

In certain embodiments, xylazine is administered topically in pharmaceutical compositions comprising about 0.01 to about 5 wt % of xylazine. For example, the pharmaceutical compositions comprise about 0.01, 0.02, 0.03, 0.033, 0.04, 0.05, 0.06, 0.066, 0.07, 0.08, 0.09, 0.1, 0.2, 0.3, 0.33, 0.4, 0.5, 0.6, 0.66, 0.7, 0.8, 0.9, 1, 1.33, 1.5, 1.66, 2, 2.33, 2.5, 2.66, 3, 3.33, 3.5, 3.66, 4, 4.33, 4.5, 4.66 or 5 wt % of xylazine. In certain embodiments, the pharmaceutical compositions comprise 0.033, 0.1, 0.33 or 1 wt % of xylazine. In a preferred embodiment, the pharmaceutical composition comprises 0.1 wt % of xylazine. In another preferred embodiment, the pharmaceutical composition comprises 0.33 wt % of xylazine. In another preferred embodiment, the pharmaceutical composition comprises 1 wt % of xylazine.

In certain embodiments, the topically administered alpha-2 adrenoreceptor agonist is the only, or sole, active agent being administered to treat pruritus. In other embodiments, the topically administered alpha-2 adrenoreceptor agonist is administered in combination with at least one additional active agent.

In certain embodiments, the topically administered alpha-2 adrenoreceptor agonist that is apraclonidine, brimonidine, clonidine, detomidine, dexmedetomidine, guanabenz, lofexidine, medetomidine, romifidine or xylazine, is the only, or sole, active agent being administered to treat pruritus. In other embodiments, the topically administered alpha-2 adrenoreceptor agonist that is apraclonidine, brimonidine, clonidine, detomidine, dexmedetomidine, guanabenz, lofexidine, medetomidine, romifidine or xylazine, is administered in combination with at least one additional active agent.

In certain embodiments, the additional active agent is also administered topically, either in a combined, or as separate, pharmaceutical compositions. In other embodiments, the additional active agent is administered orally or parenterally. Examples of parenteral administration include intravenous, intramuscular, subcutaneous, rectal, sublingual, buccal, inhaled and intrathecal administrations.

Examples of additional active agents include corticosteroids, doxepine, tacrolimus, pimecrolimus, pramoxine, lidocaine, prilocaine, ketamine, amitriptyline, capsaicin, menthol, camphor, strontium, tofacitinib, crisaborole, N-palmitoylethanolamine, antihistamines, SNRIs, SSRIs, naltrexone, butophanol, nalfurafine, gabapentin, pregabalin, aprepitant, thalidomide, lenalidomide, ursodeoxycholic acid, rifampin, cholestyramine, phenobarbital, Botulinum toxin A, naloxone, ASN008, SNA-125, TS-022, KPL-716 and orvepitant.

Examples of corticosteroids include alclometasone, amcinonide, beclometasone, betamethasone, budesonide, ciclesonide, clobetasol, clobetasone, clocortolone, cortisone, desonide, desoximetasone, dexamethasone, diflorasone, fluocinolone, fluocortolone, fluprednidene, flurandrenolide, fluticasone, halcinonide, halobetasol, halometasone, hydrocortisone, mometasone, methylprednisolone, prednicarbate, prednisolone, prednisone, tixocortol, triamcinolone and mometasone.

Examples of anti-histamines include acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, rupatadine, tripelennamine and triprolidine.

Examples of SNRIs include venlafaxine, duloxetine, milnacipran, mirtazapine and levomilnacipran.

Examples of SSRIs include fluoxetine, fluvoxamine, paroxetine, sertraline, citalopram and escitalopram.

In certain embodiments, the alpha-2 adrenoreceptor agonist is topically administered once daily to treat pruritus. In other embodiments, the alpha-2 adrenoreceptor agonist is topically administered twice daily to treat pruritus. In other embodiments, the alpha-2 adrenoreceptor agonist is topically administered three times daily to treat pruritus.

In certain embodiments, the alpha-2 adrenoreceptor agonist that is apraclonidine, brimonidine, clonidine, detomidine, dexmedetomidine, guanabenz, lofexidine, medetomidine, romifidine or xylazine, is topically administered once daily to treat pruritus. In other embodiments, the alpha-2 adrenoreceptor agonist that is apraclonidine, brimonidine, clonidine, detomidine, dexmedetomidine, guanabenz, lofexidine, medetomidine, romifidine or xylazine, is topically administered twice daily to treat pruritus. In other embodiments, the alpha-2 adrenoreceptor agonist that is apraclonidine, brimonidine, clonidine, detomidine, dexmedetomidine, guanabenz, lofexidine, medetomidine, romifidine or xylazine, is topically administered three times daily to treat pruritus.

In certain embodiments, apraclonidine is topically administered once daily to treat pruritus. In other embodiments, apraclonidine is topically administered twice daily to treat pruritus. In other embodiments, apraclonidine is topically administered three times daily to treat pruritus.

In certain embodiments, brimonidine is topically administered once daily to treat pruritus. In other embodiments, brimonidine is topically administered twice daily to treat pruritus. In other embodiments, brimonidine is topically administered three times daily to treat pruritus.

In certain embodiments, clonidine is topically administered once daily to treat pruritus. In other embodiments, clonidine is topically administered twice daily to treat pruritus. In other embodiments, clonidine is topically administered three times daily to treat pruritus.

In certain embodiments, detomidine is topically administered once daily to treat pruritus. In other embodiments, detomidine is topically administered twice daily to treat pruritus. In other embodiments, detomidine is topically administered three times daily to treat pruritus.

In certain embodiments, dexmedetomidine is topically administered once daily to treat pruritus. In other embodiments, dexmedetomidine is topically administered twice daily to treat pruritus. In other embodiments, dexmedetomidine is topically administered three times daily to treat pruritus.

In certain embodiments, guanabenz is topically administered once daily to treat pruritus. In other embodiments, guanabenz is topically administered twice daily to treat pruritus. In other embodiments, guanabenz is topically administered three times daily to treat pruritus.

In certain embodiments, lofexidine is topically administered once daily to treat pruritus. In other embodiments, lofexidine is topically administered twice daily to treat pruritus. In other embodiments, lofexidine is topically administered three times daily to treat pruritus.

In certain embodiments, medetomidine is topically administered once daily to treat pruritus. In other embodiments, medetomidine is topically administered twice daily to treat pruritus. In other embodiments, medetomidine is topically administered three times daily to treat pruritus.

In certain embodiments, romifidine is topically administered once daily to treat pruritus. In other embodiments, romifidine is topically administered twice daily to treat pruritus. In other embodiments, romifidine is topically administered three times daily to treat pruritus.

In certain embodiments, xylazine is topically administered once daily to treat pruritus. In other embodiments, xylazine is topically administered twice daily to treat pruritus. In other embodiments, xylazine is topically administered three times daily to treat pruritus.

Alpha-2 adrenoreceptor agonists can be administered topically to treat pruritus in the form of a pharmaceutical composition. Examples of pharmaceutical compositions for the topical administration of an alpha-2 adrenoreceptor agonist to treat pruritus include gels, creams, ointments, emulsions, emu-gels, foams, suspensions and spray-patches. In a preferred embodiment, the alpha-2 adrenoreceptor agonist is topically administered in the form of a gel.

Apraclonidine can be administered topically to treat pruritus in the form of a pharmaceutical composition. Examples of pharmaceutical compositions for the topical administration of apraclonidine to treat pruritus include gels, creams, ointments, emulsions, emu-gels, foams, suspensions and spray-patches. In a preferred embodiment, apraclonidine is topically administered in the form of a gel.

Brimonidine can be administered topically to treat pruritus in the form of a pharmaceutical composition. Examples of pharmaceutical compositions for the topical administration of brimonidine to treat pruritus include gels, creams, ointments, emulsions, emu-gels, foams, suspensions and spray-patches. In a preferred embodiment, brimonidine is topically administered in the form of a gel.

Clonidine can be administered topically to treat pruritus in the form of a pharmaceutical composition. Examples of pharmaceutical compositions for the topical administration of clonidine to treat pruritus include gels, creams, ointments, emulsions, emu-gels, foams, suspensions and spray-patches. In a preferred embodiment, clonidine is topically administered in the form of a gel.

Detomidine can be administered topically to treat pruritus in the form of a pharmaceutical composition. Examples of pharmaceutical compositions for the topical administration of detomidine to treat pruritus include gels, creams, ointments, emulsions, emu-gels, foams, suspensions and spray-patches. In a preferred embodiment, detomidine is topically administered in the form of a gel.

Dexmedetomidine can be administered topically to treat pruritus in the form of a pharmaceutical composition. Examples of pharmaceutical compositions for the topical administration of dexmedetomidine to treat pruritus include gels, creams, ointments, emulsions, emu-gels, foams, suspensions and spray-patches. In a preferred embodiment, dexmedetomidine is topically administered in the form of a gel.

Guanabenz can be administered topically to treat pruritus in the form of a pharmaceutical composition. Examples of pharmaceutical compositions for the topical administration of guanabenz to treat pruritus include gels, creams, ointments, emulsions, emu-gels, foams, suspensions and spray-patches. In a preferred embodiment, guanabenz is topically administered in the form of a gel.

Lofexidine can be administered topically to treat pruritus in the form of a pharmaceutical composition. Examples of pharmaceutical compositions for the topical administration of lofexidine to treat pruritus include gels, creams, ointments, emulsions, emu-gels, foams, suspensions and spray-patches. In a preferred embodiment, lofexidine is topically administered in the form of a gel.

Medetomidine can be administered topically to treat pruritus in the form of a pharmaceutical composition. Examples of pharmaceutical compositions for the topical administration of medetomidine to treat pruritus include gels, creams, ointments, emulsions, emu-gels, foams, suspensions and spray-patches. In a preferred embodiment, medetomidine is topically administered in the form of a gel.

Romifidine can be administered topically to treat pruritus in the form of a pharmaceutical composition. Examples of pharmaceutical compositions for the topical administration of romifidine to treat pruritus include gels, creams, ointments, emulsions, emu-gels, foams, suspensions and spray-patches. In a preferred embodiment, romifidine is topically administered in the form of a gel.

Xylazine can be administered topically to treat pruritus in the form of a pharmaceutical composition. Examples of pharmaceutical compositions for the topical administration of xylazine to treat pruritus include gels, creams, ointments, emulsions, emu-gels, foams, suspensions and spray-patches. In a preferred embodiment, xylazine is topically administered in the form of a gel.

In one embodiment, the present invention relates to a method of treating pruritus comprising the topical administration of a 0.033 wt % gel of an alpha-2 adrenoreceptor agonist. In another embodiment, the present invention relates to a method of treating pruritus comprising the topical administration of a 0.1 wt % gel of an alpha-2 adrenoreceptor agonist. In another embodiment, the present invention relates to a method of treating pruritus comprising the topical administration of a 0.33 wt % gel of an alpha-2 adrenoreceptor agonist. In another embodiment, the present invention relates to a method of treating pruritus comprising the topical administration of a 1 wt % gel of an alpha-2 adrenoreceptor agonist. In one embodiment, the administration is once daily. In another embodiment, the administration is twice daily. In yet another embodiment, the administration is three times a day.

In one embodiment, the present invention relates to a method of treating pruritus comprising the topical administration of a 0.033 wt % gel of apraclonidine. In another embodiment, the present invention relates to a method of treating pruritus comprising the topical administration of a 0.1 wt % gel of apraclonidine. In another embodiment, the present invention relates to a method of treating pruritus comprising the topical administration of a 0.33 wt % gel of apraclonidine. In another embodiment, the present invention relates to a method of treating pruritus comprising the topical administration of a 1 wt % gel of apraclonidine. In one embodiment, the administration is once daily. In another embodiment, the administration is twice daily. In yet another embodiment, the administration is three times a day.

In one embodiment, the present invention relates to a method of treating pruritus comprising the topical administration of a 0.033 wt % gel of brimonidine. In another embodiment, the present invention relates to a method of treating pruritus comprising the topical administration of a 0.1 wt % gel of brimonidine. In another embodiment, the present invention relates to a method of treating pruritus comprising the topical administration of a 0.33 wt % gel of brimonidine. In another embodiment, the present invention relates to a method of treating pruritus comprising the topical administration of a 1 wt % gel of brimonidine. In one embodiment, the administration is once daily. In another embodiment, the administration is twice daily. In yet another embodiment, the administration is three times a day.

In one embodiment, the present invention relates to a method of treating pruritus comprising the topical administration of a 0.033 wt % gel of clonidine. In another embodiment, the present invention relates to a method of treating pruritus comprising the topical administration of a 0.1 wt % gel of clonidine. In another embodiment, the present invention relates to a method of treating pruritus comprising the topical administration of a 0.33 wt % gel of clonidine. In another embodiment, the present invention relates to a method of treating pruritus comprising the topical administration of a 1 wt % gel of clonidine. In one embodiment, the administration is once daily. In another embodiment, the administration is twice daily. In yet another embodiment, the administration is three times a day.

In one embodiment, the present invention relates to a method of treating pruritus comprising the topical administration of a 0.033 wt % gel of detomidine. In another embodiment, the present invention relates to a method of treating pruritus comprising the topical administration of a 0.1 wt % gel of detomidine. In another embodiment, the present invention relates to a method of treating pruritus comprising the topical administration of a 0.33 wt % gel of detomidine. In another embodiment, the present invention relates to a method of treating pruritus comprising the topical administration of a 1 wt % gel of detomidine. In one embodiment, the administration is once daily. In another embodiment, the administration is twice daily.

In one embodiment, the present invention relates to a method of treating pruritus comprising the topical administration of a 0.033 wt % gel of medetomidine. In another embodiment, the present invention relates to a method of treating pruritus comprising the topical administration of a 0.1 wt % gel of medetomidine. In another embodiment, the present invention relates to a method of treating pruritus comprising the topical administration of a 0.33 wt % gel of medetomidine. In another embodiment, the present invention relates to a method of treating pruritus comprising the topical administration of a 1 wt % gel of medetomidine. In one embodiment, the administration is once daily. In another embodiment, the administration is twice daily. In yet another embodiment, the administration is three times a day.

In one embodiment, the present invention relates to a method of treating pruritus comprising the topical administration of a 0.033 wt % gel of dexmedetomidine. In another embodiment, the present invention relates to a method of treating pruritus comprising the topical administration of a 0.1 wt % gel of dexmedetomidine. In another embodiment, the present invention relates to a method of treating pruritus comprising the topical administration of a 0.33 wt % gel of dexmedetomidine. In another embodiment, the present invention relates to a method of treating pruritus comprising the topical administration of a 1 wt % gel of dexmedetomidine. In one embodiment, the administration is once daily. In another embodiment, the administration is twice daily. In yet another embodiment, the administration is three times a day.

In one embodiment, the present invention relates to a method of treating pruritus comprising the topical administration of a 0.033 wt % gel of guanabenz. In another embodiment, the present invention relates to a method of treating pruritus comprising the topical administration of a 0.1 wt % gel of guanabenz. In another embodiment, the present invention relates to a method of treating pruritus comprising the topical administration of a 0.33 wt % gel of guanabenz. In another embodiment, the present invention relates to a method of treating pruritus comprising the topical administration of a 1 wt % gel of guanabenz. In one embodiment, the administration is once daily. In another embodiment, the administration is twice daily. In yet another embodiment, the administration is three times a day.

In one embodiment, the present invention relates to a method of treating pruritus comprising the topical administration of a 0.033 wt % gel of lofexidine. In another embodiment, the present invention relates to a method of treating pruritus comprising the topical administration of a 0.1 wt % gel of lofexidine. In another embodiment, the present invention relates to a method of treating pruritus comprising the topical administration of a 0.33 wt % gel of lofexidine. In another embodiment, the present invention relates to a method of treating pruritus comprising the topical administration of a 1 wt % gel of lofexidine. In another embodiment, the present invention relates to a method of treating pruritus comprising the topical administration of a 2 wt % gel of lofexidine. In one embodiment, the administration is once daily. In another embodiment, the administration is twice daily. In yet another embodiment, the administration is three times a day.

In one embodiment, the present invention relates to a method of treating pruritus comprising the topical administration of a 0.033 wt % gel of romifidine. In another embodiment, the present invention relates to a method of treating pruritus comprising the topical administration of a 0.1 wt % gel of romifidine. In another embodiment, the present invention relates to a method of treating pruritus comprising the topical administration of a 0.33 wt % gel of romifidine. In another embodiment, the present invention relates to a method of treating pruritus comprising the topical administration of a 1 wt % gel of romifidine. In one embodiment, the administration is once daily. In another embodiment, the administration is twice daily. In yet another embodiment, the administration is three times a day.

In one embodiment, the present invention relates to a method of treating pruritus comprising the topical administration of a 0.033 wt % gel of xylazine. In another embodiment, the present invention relates to a method of treating pruritus comprising the topical administration of a 0.1 wt % gel of xylazine. In another embodiment, the present invention relates to a method of treating pruritus comprising the topical administration of a 0.33 wt % gel of xylazine. In another embodiment, the present invention relates to a method of treating pruritus comprising the topical administration of a 1 wt % gel of xylazine. In one embodiment, the administration is once daily. In another embodiment, the administration is twice daily. In yet another embodiment, the administration is three times a day.

Topical administration of the pharmaceutical compositions to a subject can result in a blood plasma concentration in the subject that is less than that required to achieve a systemic therapeutic effect of the alpha-2 adrenoreceptor agonist. Preferably, the topical administration can continue for weeks, months, or longer while maintaining a sub-therapeutic systemic blood plasma concentration and with minimal or no medically relevant effect outside of that bodily region, or simply minimal or no medically relevant systemic effect.

In certain embodiments, the pharmaceutical compositions of the present invention provide prolonged, substantially non-systemic treatment for pruritus. The period of time over which the pharmaceutical compositions can provide treatment for pruritus is up to 24 hours when topically applied once a day. In certain embodiments, the pharmaceutical compositions are preferably applied twice per day, and in such instances the treatment of pruritus that is provided by a first of the two topical administrations has a duration that lasts until the second topical administration, and the second daily topical administration has a duration that lasts until the following day's first topical administration. As used herein, “substantially non-systemic” refers to a treatment effect that is localized to the bodily region (for example, body part) to which the pharmaceutical compositions is topically applied, with a minimal or no medically relevant effect outside of that bodily region, or simply no minimal or no medically relevant systemic effect.

In certain embodiments, the pharmaceutical compositions of the present invention comprise an alpha-2 adrenoreceptor agonist and provide prolonged, substantially non-systemic treatment for pruritus. In some embodiments, the alpha-2 adrenoreceptor agonist is apraclonidine, brimonidine, clonidine, detomidine, dexmedetomidine, guanabenz, lofexidine, medetomidine, romifidine or xylazine.

Examples of gels, creams, ointments, emulsions, emu-gels, foams, suspensions and spray-patches of alpha-2 adrenoreceptor agonists are described in U.S. Pat. Nos. 6,534,048, 8,026,266, 8,231,885, 8,513,247, WO9221338, WO2009158477, WO2011085162, WO2012083397, WO2018129313, WO2020012415 and U.S. provisional application 62/964,191 all of which are incorporated herein by reference in their entirety.

Pharmaceutical compositions of alpha-2 adrenoreceptor agonists for topical administration can also include a carrier that is suitable for topical administration to a subject's skin. The carrier may include, for example, a solubilizer, a buffer, or both. The carrier can also be a mixture of a hydrophilic phase member and a hydrophobic phase member. As described below, the formulation may also include one or more additional components in order to produce the topical form, such as thickening or gelling agents, preservatives, antioxidants, permeation enhancers, emulsifying agents, emollients, or humectants.

Examples of solubilizers include alcohols, such as sugar alcohols, diols, polyols, or polyether alcohols, fatty acids, organic solvents, waxes, oils, poloxamers, cyclodextrins, or any combination thereof. For example, the solubilizer may be glycerol, polyethylene glycol (such as PEG 3350), propylene glycol, poloxamer 124, poloxamer 407, Labrasol® (caprylocaproyl polyoxyl-8 glycerides), Kleptose® HPB, Captisol® sulfobutylether β-cyclodextrin, or any combination thereof. In some embodiments, the solubilizer is glycerol, propylene glycol, polyethylene glycol, or any combination thereof. For example, the water-miscible solubilizer may include both glycerol and propylene glycol.

Examples of hydrophilic phase members include water, glycerol, polypropylene glycol, polyethylene glycol, ethanol, benzyl alcohol, 1,3-propanediol, 1,2-pentanediol, propylene carbonate, 2-(2-ethoxyethoxy)ethanol, dimethyl isosorbide, tetraglycol, pyrrolidone, dimethylacetamide, caprylocaproyl polyoxyl-8 glycerides, hexylene glycol, butylene glycol, or any combination thereof. The hydrophilic phase member may also comprise an aqueous buffer solution. For example, the hydrophilic phase member may comprise 0.01 to 1.0M citrate, phosphate, Tris, carbonate, succinate, tartrate, borate, imidazole, maleate, or phthalate buffer at pH 4.5-9.0.

Examples of hydrophobic phase members include aromatic hydrocarbons, alkane, cycloalkanes, alkynes, terpenes, organic oils, mineral oils, or any combination thereof. Exemplary hydrophilic phase members include mineral oil, isopropyl isostearate, isostearyl isostearate, alkyl benzoate, butyl stearate, diisopropyl adipate, diethylhexyl adipate, caprilic/capric triglyceride, isocetyl stearate, isopropyl myristate, isopropyl palmitate, lauryl lactate, myristil myristate, ethylhexyl cocoate, ethylhexyl palmiatate, ethylhexyl pelagronate, ethylhexyl stearate, diethylhexyl succinate, propylene glycol dicaprylate/dicaprate, PPG-2 myristyl ether propionate, pentaerythrityl tetracaprylate/caprate, pentaerythrityl tetraisostearate, PEG 2 stearyl ether, steareth-21, and isotridecyl isononanoate. An exemplary genus of hydrophobic phase members is medium chain triglycerides. Further hydrophobic phase members that represent fatty acid esters are disclosed in U.S. Pub. No. 2012/0201863, the entire contents of which are incorporated herein by reference.

Examples of thickening or gelling agents can include hydroxymethyl cellulose, hydroxyethyl cellulose, hydroxypropyl cellulose, hydroxypropylmethyl cellulose, xanthan gum, carbomers (acrylates and acrylic acid and its derivatives polymers, such as Carbopol® 980 (crosslinked polyacrylate polymer)), povidones (e.g., polyvinylpyrrolidone), Poloxamers, Polyamide-3 (e.g., OleoCraft™ HP33), glyceryl polyacrylates and other appropriate agents and combinations thereof.

Examples of preservatives can include benzalkonium chloride, parabens, sorbic acid and its salts, benzoic acid and its salts, cetrimonium bromide and chloride salts, phenoxyethanol, and other agents.

Examples of antioxidants can include sodium metabisulfite, ascorbic acid, tocopheryl acetate (for purely aqueous formulations), and BHT or BHA (for hydrophobic formulations).

Examples of permeation enhancers can include Transcutol® P (highly purified diethylene glycol monoethyl ether EP/NF), Laurocapram (Azone) or dimethylisosorbide (DMI).

Examples of emulsifying agents can include Tweens, Spans, poloxamers (124, 407, 188), Brij S2 and Brij 721, Crodex M (cetearyl alcohol and potassium cetyl Phosphate), Crodafos CES (cetearyl alcohol and dicetyl phosphate and Ceteth-10 phosphate (Crodafos CES), Cithrol DPHS (PEG 30 Dipolyhydroxystearate), cyclopentasiloxane, or macrogol hydroxystearate.

Examples of emollients can include, but are not limited to, Migliol 810 or 812 (caprylic-capric triglycerides), Isoporpyl Isostearate (Crodamol IPIS), Isostearyl Isostearate (Crodamol ISIS), PPG-11 Stearyl Ether (Arlamol PS HE), Macrogol 6 Glycerol Caprylocaprate (Glycerox 767HC), or Labrasol® (caprylocaproyl polyoxyl-8 glycerides).

Examples of humectants can include, but are not limited to, glycerin, propylene glycol, 1,3-propanediol, or 1,2-pentanediol.

In certain embodiments, the topical pharmaceutical compositions comprise 0.01 to 5 wt % apraclonidine hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % apraclonidine hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % apraclonidine hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % apraclonidine hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % apraclonidine hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % apraclonidine hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % brimonidine hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % brimonidine hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % brimonidine hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % brimonidine hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % brimonidine hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % brimonidine hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % clonidine hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % clonidine hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % clonidine hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % clonidine hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % clonidine hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % clonidine hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % detomidine hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % detomidine hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % detomidine hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % detomidine hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % detomidine hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % detomidine hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % medetomidine hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % medetomidine hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % medetomidine hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % medetomidine hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % medetomidine hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % medetomidine hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % dexmedetomidine hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % dexmedetomidine hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % dexmedetomidine hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % dexmedetomidine hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % dexmedetomidine hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % dexmedetomidine hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % guanabenz hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % guanabenz hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % guanabenz hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % guanabenz hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % guanabenz hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % guanabenz hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % lofexidine hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % lofexidine hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % lofexidine hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % lofexidine hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % lofexidine hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % lofexidine hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % romifidine hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % romifidine hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % romifidine hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % romifidine hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % romifidine hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % romifidine hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % xylazine hydrochloride, glycerine, propylene glycol, a gelling agent, and a buffer that is effective to maintain the pharmaceutical composition at pH about 4.5 to about 8.2. In some embodiments, the topical pharmaceutical compositions comprise 0.01 to 3 wt % xylazine hydrochloride, glycerine, propylene glycol, a cellulose gelling agent, and a buffer that is effective to maintain the pharmaceutical composition at pH about 4.5 to about 6. In some other embodiments, the topical pharmaceutical compositions comprise 0.05 to 3 wt % xylazine hydrochloride, glycerine, propylene glycol, a cellulose gelling agent, and a buffer that is effective to maintain the pharmaceutical composition at pH about 5 to about 6. In yet other embodiments, the topical pharmaceutical compositions comprise 0.1 to 2 wt % xylazine hydrochloride, glycerine, propylene glycol, a cellulose gelling agent, and a buffer that is effective to maintain the pharmaceutical composition at pH about 5 to about 5.5. In still other embodiments, the topical pharmaceutical compositions comprise 0.1 to 1 wt % xylazine hydrochloride, glycerine, propylene glycol, hydroxy ethyl cellulose, and a buffer that is effective to maintain the pharmaceutical composition at pH about 5 to about 5.5. In yet other embodiments, the topical pharmaceutical compositions comprise 0.1 to 1 wt % xylazine hydrochloride, glycerine, propylene glycol, hydroxy ethyl cellulose, and a buffer that is effective to maintain the pharmaceutical composition at pH about 5.2 to about 5.5. Any of these embodiments may further comprise a preservative.

In certain embodiments, the topical pharmaceutical compositions comprise 0.01 to 5 wt % brimonidine, 0.05 to 0.20 wt % of at least one preservative, 0.80 to 1.50 wt % of a thickening or gelling agent, 9.0 to 13.0 wt % of an inert excipient and have a pH of 4.5 to 7.5. In certain embodiments, the topical pharmaceutical compositions comprise 0.01 to 5 wt % brimonidine, 0.05 to 0.20 wt % methylparaben, 0.80 to 1.50 wt % carbomer, 9.0 to 13.0 wt % total polyol and have a pH of 4.5 to 7.5.

In certain embodiments, the topical pharmaceutical compositions comprise 0.01 to 5 wt % medetomidine hydrochloride and optionally 0.01 to 65 wt % of thickening or gelling agents, 0.001 to 20 wt % of preservatives and/or 0.01 to 5 wt % of buffers.

In certain embodiments, the topical pharmaceutical compositions comprise 0.01 to 5 wt wt % dexmedetomidine hydrochloride and optionally 0.01 to 65 wt % of thickening or gelling agents, 0.001 to 20 wt % of preservatives and/or 0.01 to 5 wt % of buffers.

In certain embodiments, the topical pharmaceutical compositions comprise 0.1 to 50% lofexidine, 1 to 50 wt % water, 10 to 98 wt % propylene glycol and 1 to 10 wt % hydroxypropyl ethylcellulose. In other embodiments, the topical pharmaceutical compositions comprise 0.1 to 50% lofexidine, 5 to 30 wt % water, 30 to 70 wt % propylene glycol, 1 to 5 wt % hydroxypropyl ethylcellulose and 0.01 to 5 wt % preservative. In other embodiments, the topical pharmaceutical compositions comprise 0.1 to 50% lofexidine, 10 to 98 wt % propylene glycol and 1 to 10 wt % hydroxypropyl methylcellulose.

In certain embodiments, the topical pharmaceutical compositions comprise at least about 0.01 weight percent of an alpha-2 adrenoreceptor agonist, based on the total weight of the composition. In some embodiments, the alpha-2 adrenoreceptor agonist is present in an amount in the range of about 0.01 to about 0.5 weight percent. In some other embodiments, the alpha-2 adrenoreceptor agonist is present in an amount in the range of about 0.01 to about 0.25 weight percent. In yet another embodiment, the alpha-2 adrenoreceptor agonist is present in an amount in the range of about 0.01 to about 0.075 weight percent.

As discussed herein, all weight percentage of alpha-2 adrenoreceptor agonist is calculated based on the weight of the topical pharmaceutical composition, e.g., a gel.

In certain embodiments, the topical pharmaceutical compositions comprise at least about 0.01 weight percent of apraclonidine, based on the total weight of the composition. In some embodiments, apraclonidine is present in an amount in the range of about 0.01 to about 0.5 weight percent. In some other embodiments, apraclonidine is present in an amount in the range of about 0.01 to about 0.25 weight percent. In yet another embodiment, apraclonidine is present in an amount in the range of about 0.01 to about 0.075 weight percent. As discussed herein, all weight percentage of apraclonidine is calculated based on the weight of the topical pharmaceutical composition, e.g., a gel.

In certain embodiments, the topical pharmaceutical compositions comprise at least about 0.01 weight percent of brimonidine, based on the total weight of the composition. In some embodiments, brimonidine is present in an amount in the range of about 0.01 to about 0.5 weight percent. In some other embodiments, brimonidine is present in an amount in the range of about 0.01 to about 0.25 weight percent. In yet another embodiment, brimonidine is present in an amount in the range of about 0.01 to about 0.075 weight percent. As discussed herein, all weight percentage of brimonidine is calculated based on the weight of the topical pharmaceutical composition, e.g., a gel.

In certain embodiments, the topical pharmaceutical compositions comprise at least about 0.01 weight percent of clonidine, based on the total weight of the composition. In some embodiments, clonidine is present in an amount in the range of about 0.01 to about 0.5 weight percent. In some other embodiments, clonidine is present in an amount in the range of about 0.01 to about 0.25 weight percent. In yet another embodiment, clonidine is present in an amount in the range of about 0.01 to about 0.075 weight percent. As discussed herein, all weight percentage of clonidine is calculated based on the weight of the topical pharmaceutical composition, e.g., a gel.

In certain embodiments, the topical pharmaceutical compositions comprise at least about 0.01 weight percent of detomidine, based on the total weight of the composition. In some embodiments, detomidine is present in an amount in the range of about 0.01 to about 0.5 weight percent. In some other embodiments, detomidine is present in an amount in the range of about 0.01 to about 0.25 weight percent. In yet another embodiment, detomidine is present in an amount in the range of about 0.01 to about 0.075 weight percent. As discussed herein, all weight percentage of detomidine is calculated based on the weight of the topical pharmaceutical composition, e.g., a gel.

In certain embodiments, the topical pharmaceutical compositions comprise at least about 0.01 weight percent of medetomidine, based on the total weight of the composition. In some embodiments, medetomidine is present in an amount in the range of about 0.01 to about 0.5 weight percent. In some other embodiments, medetomidine is present in an amount in the range of about 0.01 to about 0.25 weight percent. In yet another embodiment, medetomidine is present in an amount in the range of about 0.01 to about 0.075 weight percent. As discussed herein, all weight percentage of medetomidine is calculated based on the weight of the topical pharmaceutical composition, e.g., a gel.

In certain embodiments, the topical pharmaceutical compositions comprise at least about 0.01 weight percent of dexmedetomidine, based on the total weight of the composition. In some embodiments, dexmedetomidine is present in an amount in the range of about 0.01 to about 0.5 weight percent. In some other embodiments, dexmedetomidine is present in an amount in the range of about 0.01 to about 0.25 weight percent. In yet another embodiment, dexmedetomidine is present in an amount in the range of about 0.01 to about 0.075 weight percent. As discussed herein, all weight percentage of dexmedetomidine is calculated based on the weight of the topical pharmaceutical composition, e.g., a gel.

In certain embodiments, the topical pharmaceutical compositions comprise at least about 0.01 weight percent of guanabenz, based on the total weight of the composition. In some embodiments, guanabenz is present in an amount in the range of about 0.01 to about 0.5 weight percent. In some other embodiments, guanabenz is present in an amount in the range of about 0.01 to about 0.25 weight percent. In yet another embodiment, guanabenz is present in an amount in the range of about 0.01 to about 0.075 weight percent. As discussed herein, all weight percentage of guanabenz is calculated based on the weight of the topical pharmaceutical composition, e.g., a gel.

In certain embodiments, the topical pharmaceutical compositions comprise at least about 0.01 weight percent of lofexidine, based on the total weight of the composition. In some embodiments, lofexidine is present in an amount in the range of about 0.01 to about 0.5 weight percent. In some other embodiments, lofexidine is present in an amount in the range of about 0.01 to about 0.25 weight percent. In yet another embodiment, lofexidine s present in an amount in the range of about 0.01 to about 0.075 weight percent. As discussed herein, all weight percentage of lofexidine is calculated based on the weight of the topical pharmaceutical composition, e.g., a gel.

In certain embodiments, the topical pharmaceutical compositions comprise at least about 0.01 weight percent of romifidine, based on the total weight of the composition. In some embodiments, romifidine is present in an amount in the range of about 0.01 to about 0.5 weight percent. In some other embodiments, romifidine is present in an amount in the range of about 0.01 to about 0.25 weight percent. In yet another embodiment, romifidine is present in an amount in the range of about 0.01 to about 0.075 weight percent. As discussed herein, all weight percentage of romifidine is calculated based on the weight of the topical pharmaceutical composition, e.g., a gel.

In certain embodiments, the topical pharmaceutical compositions comprise at least about 0.01 weight percent of xylazine, based on the total weight of the composition. In some embodiments, xylazine is present in an amount in the range of about 0.01 to about 0.5 weight percent. In some other embodiments, xylazine is present in an amount in the range of about 0.01 to about 0.25 weight percent. In yet another embodiment, xylazine is present in an amount in the range of about 0.01 to about 0.075 weight percent. As discussed herein, all weight percentage of xylazine is calculated based on the weight of the topical pharmaceutical composition, e.g., a gel.

The pharmaceutical compositions may include a volatile solvent that at least partially evaporates from the skin surface following application. For example, in certain embodiments, the buffer component is aqueous, and the water that is contained within the aqueous buffer represents the volatile solvent. The portion of the formulation that remains following at least partial evaporation can be referred to as the “nonvolatile” or “residual” phase, and the volatile element(s) of the formulation that evaporate from the skin surface represents the “volatile” phase.

In certain embodiments, the pharmaceutical compositions may include an inert excipient that assists with increasing the concentration of the alpha-2 adrenoreceptor agonist in the residual phase following topical application. In effect, such excipients can cause “salting out” of the agonist, or salt thereof, from the other components of the residual phase, which can have the effect of increasing the activity of the agonist, or salt thereof, on the surface of the subject's skin and promote permeability of the drug through the skin. Such inert excipients can include, for example, a polyol or simple sugar, such as sucrose, dextrose, trehalose, mannitol, sorbitol, or xylitol.

In certain embodiments, pharmaceutical compositions may comprise a foam. Foams according to the present disclosure may include a hydrophobic phase member that comprises, for example, a medium chain triglyceride, mineral oil, or both. The hydrophilic phase member in the foams may include, for example, one or more of propylene glycol, hexylene glycol, 1,3-propanediol, 1,2-pentanediol or water.

The pharmaceutical compositions may comprise a cream. In cream formulations, the hydrophobic phase member may comprise, for example, mineral oil, isopropyl isostearate, isostearyl isostearate, alkyl benzoate, butyl stearate, diisopropyl adipate, diethylhexyl adipate, caprilic/capric triglyceride, isocetyl stearate, isopropyl myristate, isopropyl palmitate, lauryl lactate, myristil myristate, ethylhexyl cocoate, ethylhexyl palmiatate, ethylhexyl pelagronate, ethylhexyl stearate, diethylhexyl succinate, propylene glycol dicaprylate/dicaprate, PPG-2 myristyl ether propionate, pentaerythrityl tetracaprylate/caprate, pentaerythrityl tetraisostearate, isotridecyl isononanoate, or any combination thereof. The hydrophilic phase member may be, for example, glycerol, propylene glycol, water, 1,3-propanediol, 1,2-pentanediol, hexylene glycol, butylene glycol, or any combination thereof. Cream formulations may further comprise a fatty alcohol, an ester of a fatty alcohol, or both, an emulsifier, an emollient, or any combination thereof, including each of these components.

In accordance with the presently disclosed methods, the topical administration may be performed using conventional techniques. For example, the administration may be performed by delivering an aliquot of the pharmaceutical composition to a physician's or subject's hand, which is used to smear and then rub the pharmaceutical composition onto an area of skin on the body part for which treatment is desired. In the case of a spray patch, the pharmaceutical composition may be sprayed using any suitable mechanism, such as an aerosol, mister, spray bottle, or the like. The pharmaceutical composition may be topically administered in the chosen manner on a once-daily, twice-daily or three-times daily basis. When the method comprises applying the composition on a twice-daily basis, appropriate temporal spacing between applications should be used. For example, if the subject is awake for a 16 hour period of the day, then a first application can be performed in the morning, and a second application can be performed in the evening, for example, prior to retiring to bed.

### EXAMPLES

The following examples are set forth so as to provide those of ordinary skill in the art with a complete disclosure and description of how the methods, compositions, and components claimed herein are made and evaluated, and are intended to be purely exemplary of the invention and are not intended to limit the scope of what the inventors regard as their invention.

### Example 1: Topical Brimonidine Gels

Topical formulations containing brimonidine tartrate were prepared. The prepared formulations are described in Table 1.

| Ingredient | % (w/w) |
|---|---|
| Brimonidine tartrate | 0.45-0.55% |
| Carbomer 934P | 1.25% |
| Methylparaben | 0.2% |
| Phenoxyethanol | 0.4% |
| Glycerol | 5.5% |
| Kowet titanium dioxide | 0.0625% |
| Propylene glycol | 5.5% |
| Sodium hydroxide | Adjust to pH 4.5-6.5 |
| DI Water | QS as 100% |

### Example 2: Topical Clonidine Gels

Topical formulations containing clonidine HCl were prepared. The prepared formulations are described in Table 2.

TABLE 2

| Ingredient | % (w/w) |
|---|---|
| Clonidine hydrochloride USP | 0.1% |
| Benzyl alcohol NF | 1.0% |
| Carbopol ® 980 NF | 0.6% |
| Sodium hydroxide NF | Adjust to pH 8 |
| Hydrochloric acid NF | Adjust to pH 8 (if necessary) |
| Purified water USP | Qs ad 100% |

### Example 3: Topical Detomidine Gels

Topical formulations containing detomidine HCl were prepared. The prepared formulations are described in Table 3.

TABLE 3

| Ingredient | % (w/w) | | | | |
|---|---|---|---|---|---|
| Detomidine HCl | 0.01% | 0.03% | 0.10% | 0.33% | 1.00% |
| Hydroxyethyl cellulose | 1.75% | 1.75% | 1.75% | 1.75% | 1.75% |
| (Natrosol 250HH) | | | | | |
| Glycerin | 0.30% | 0.30% | 0.30% | 0.33% | 3% |
| Propylene Glycol | 0.10% | 0.10% | 0.10% | 1% | 1% |
| Transcutol P | 0.10% | 0.10% | | | |
| Benzalkonium Chloride | 0.02% | 0.02% | 0.02% | 0.02% | 0.02% |
| as 0.5% sol in water | | | | | |
| Ad 100% | Tris buffer | Buffer | Buffer | Buffer | Buffer |
| | 0.05M | Phosphate | Citrate | Citrate | Citrate |
| | pH 8.2 | 0.05M | 0.05M | 0.05M | 0.2M |
| | | pH 7.2 | pH 5.5 | pH 5.5 | pH 5.2 |

### Example 4: Topical Dexmedetomidine Creams

Topical formulations containing dexmedetomidine were prepared. The prepared formulations are described in Table 4.

TABLE 4

| Ingredient | % (w/w) |
|---|---|
| Dexmedetomidine free base | 0.1% |
| Laurocapram | 3% |
| Ethanol 200 Proof | 20% |
| Purified water | 20.9% |
| Propylene glycol | 50% |
| Sorbitol | 5% |
| Hydroxypropyl cellulose | 1% |

### Example 5: Topical Lofexidine Gels

Topical formulations containing lofexidine were prepared. The prepared formulations are described in Table 5.

TABLE 5

| Ingredient | % (w/w) |
|---|---|
| Lofexidine HCl | 2 |
| Hydroxypropyl methylcellulose | 5 |
| Propylene glycol 400 | 92 |
| Benzyl alcohol | 1 |

### Example 6: Acute Itch Model

150 μl of vehicle or 0.10%, 0.33% or 1.00% topical formulations of Example 3 were administered topically over a 2 cm² area of groups of 8 mice (4 male, 4 female) for 5 consecutive days (Days 1-5). An active control group of 8 additional mice were intraperitoneally administered a single dose of U-50,488 (CAS 67198-13-4), a selective K-opioid agonist on Day 5.

On Day 4, 2 mice (1 male, 1 female) in the 1.00% topical formulation group died, all other mice completed the treatment protocol. On Day 5, 30 minutes after the administration of either vehicle, control or detomidine, 0.4 mg of chloroquine was injected subcutaneously to the mice. The number of scratching events were recorded for each of the 5 groups at 5 minute intervals from the chloroquine administration over a 30 minute total duration.

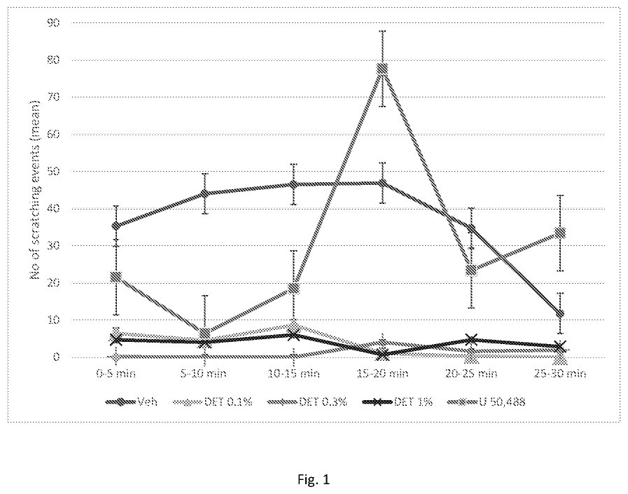

Each of the topical formulations of Example 1 were each found to reduce the number of scratching events in a statistically significant fashion over each 5 minute time point between time zero and 30 minutes. U-50,488 was found to reduce the number of scratching events in a statistically significant fashion over each 5 minute time point between time zero and 15 minutes. Apart from the death of the 2 mice on Day 4, no other adverse events were noted for any dose tested. A graphical representation of the number of scratching events over time is shown in FIG. 1.

### Example 7: Immunostaining of Pig Skin

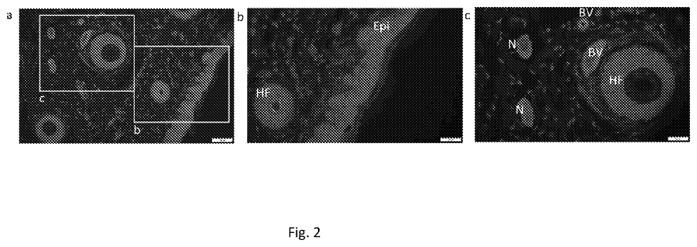

Skin biopsies were taken from the outer area of pig legs and immunostained using anti α2A adrenergic receptor polyclonal antibodies. Images were taken of the stained tissue using ×20 objective, total magnification ×200, scale bar 100 μm (Image a) or ×40 objective, total magnification ×400, scale bar 50 μm. (Images b and c). The results of the staining are shown in FIG. 2 and show positive staining in epidermis (full thickness) (Epi), hair follicles (HF), blood vessels (BV) and nerves (N) for α2A adrenergic receptors. These results identify the previously unknown presence of α2adrenegric receptors in the skin and local surrounding tissues, potentially allowing for substantially non-systemic amounts of an alpha-2 adrenoreceptor agonist to be therapeutically effective when administered topically.

### Example 8: Clinical Efficacy of Topical Detomidine

In a double blind, vehicle controlled, randomized crossover design study, the safety and efficacy of topically applied 0.1 and 1 wt % detomidine hydrochloride is assessed for the treatment of pruritus.

The study consists of a Screening Period of up to 7 days during which inclusion/exclusion criteria will be reviewed. Subjects meeting inclusion/exclusion criteria have a score of at least 5 on the 11-Point Numeric Rating Scale (NRS) for Pruritus will complete the one week Screening Period. Subjects will complete a daily diary for NRS for Pruritus scores and Sleep scores. At the end of the Screening Period, subjects who have a NRS for Pruritus score of at least 5 recorded in the diary on at least 4 of the 7 days preceding Day 0 will be eligible to continue. Baseline assessments will be recorded for vital signs, pruritic body surface area, skin integrity, PQOL, and laboratory results. The Baseline period will be followed by a 2 week Treatment Period 1 in which subjects will be randomized to 0.1 wt % detomidine hydrochloride gel or Placebo gel to be applied QD for 14 days. During the 2 week Treatment Period subjects will complete daily diaries of NRS for Pruritis scores and Sleep scores. On Day 14 subjects will return to the clinic to review diaries, adverse events (AEs), concomitant medications, and to record body surface area for pruritus, skin integrity, PQOL, and laboratory results. Subjects will then enter a Washout Period for up to 56 days until the subject again scores at least 5 on the NRS for Pruritus on 4 consecutive or 4 of the past 7 days or 56 days pass. Subjects will then enter a 2 week Treatment Period 2 during which the same procedures as Treatment Period 1 will be performed except subjects will receive the alternate treatment to that assigned in Treatment Period 1.

### Example 9: Clinical Efficacy of Topical Alpha-2 Adrenoreceptor Agonist

In a double blind, vehicle controlled, randomized crossover design study, the safety and efficacy of topically applied 0.1 and 1 wt % of an alpha-2 adrenoreceptor agonist is assessed for the treatment of pruritus.

The study consists of a Screening Period of up to 7 days during which inclusion/exclusion criteria will be reviewed. Subjects meeting inclusion/exclusion criteria have a score of at least 5 on the 11-Point Numeric Rating Scale (NRS) for Pruritus will complete the one week Screening Period. Subjects will complete a daily diary for NRS for Pruritus scores and Sleep scores. At the end of the Screening Period, subjects who have a NRS for Pruritus score of at least 5 recorded in the diary on at least 4 of the 7 days preceding Day 0 will be eligible to continue. Baseline assessments will be recorded for vital signs, pruritic body surface area, skin integrity, PQOL, and laboratory results. The Baseline period will be followed by a 2 week Treatment Period 1 in which subjects will be randomized to 0.1 wt % alpha-2 adrenoreceptor agonist or Placebo gel to be applied QD for 14 days. During the 2 week Treatment Period subjects will complete daily diaries of NRS for Pruritis scores and Sleep scores. On Day 14 subjects will return to the clinic to review diaries, adverse events (AEs), concomitant medications, and to record body surface area for pruritus, skin integrity, PQOL, and laboratory results. Subjects will then enter a Washout Period for up to 56 days until the subject again scores at least 5 on the NRS for Pruritus on 4 consecutive or 4 of the past 7 days or 56 days pass. Subjects will then enter a 2 week Treatment Period 2 during which the same procedures as Treatment Period 1 will be performed except subjects will receive the alternate treatment to that assigned in Treatment Period 1.

What is claimed:

1. A method of treating pruritus comprising the topical administration, to a subject in need thereof, of a therapeutically effective amount of an alpha-2 adrenoreceptor agonist.

2. The method of claim 1, wherein the alpha-2 adrenoreceptor agonist is apraclonidine, brimonidine, clonidine, detomidine, dexmedetomidine, guanabenz, lofexidine, medetomidine, romifidine or xylazine.

3. The method of claim 1, wherein the pruritus is acute.

4. The method of claim 1, wherein the pruritus is chronic.

5. The method of claim 1, wherein the origin of the pruritus is unknown.

6. The method of claim 1, wherein the pruritus comprises the symptom of a dermatological, neurological, psychogenic or systemic condition, or is of mixed origin.

7. The method of claim 6, wherein the dermatological condition comprises atopic dermatitis, contact dermatitis, allergic dermatitis, seborrheic dermatitis, statis dermatitis, pityriasis rubra pilaris, pityriasis rosea, acne, dermatitis herpetiformis, pemphigus vulgaris, bullous pemphigoid, lichen planus, prurigo nodularis, lichen simplex chronicus, lichen amyloidosis, urticaria, mastocytosis, polymorphous light eruption, actinic prurigo, chronic prurigo, actinic dermatitis, polymorphic eruption of pregnancy, eosinophilic folliculitis, dermatomyositis, prurigo pigmentosa, lichen sclerosus, palmoplantar pustulosis, pompholyx, idiopathic xerosis, scarring, burns, burn scars, keloid scars, hypertrophic scars, a reactive drug eruption or of an infestive or infective origin.

8. The method of claim 7, wherein the infestive origin comprises scabies, pediculosis, or an arthropod bite, or the infestive origin is fungal, parasitic, viral, or bacterial.

9. (canceled)

10. The method of claim 6, wherein the neurological condition comprises notalgia paresthetica, brachioradial pruritus, postherpetic neuralgia, stroke, small fiber neuropathy, trigeminal trophic syndrome, Creutzfeldt-Jakob disease, chemotherapy-induced neuropathy, HIV-related neuropathy or multiple sclerosis.

11. The method of claim 6, wherein the psychogenic condition comprises depression, anxiety, psychogenic excoriation, anorexia nervosa or delusional parasitosis.

12. The method of claim 6, wherein the systemic condition comprises chronic renal failure, uremic pruritus, liver disease, primary biliary cholangitis, primary biliary cirrhosis, cholestatic jaundice, hepatitis C, cholestasis of pregnancy, polycythemia vera, iron deficiency anemia, Hodgkin lymphoma, non-Hodgkin lymphoma, multiple myeloma, hematologic or lymphoproliferative disorders, primary cutaneous lymphoma, mycosis fungoides, cutaneous T cell lymphoma, malignancy, plasma cell dyscrasias, gastric carcinoid tumors, hyperthyroidism, hypothyroidism, hyperparathyroidism, haemochromatosis, celiac disease, systemic lupus erythematosus, systemic sclerosis, diabetes, carcinoid syndrome, dermatomyositis, scleroderma, Sjögren's syndrome, linear immunoglobulin A (IgA) disease, graft-versus-host disease, Darier disease, Hailey-Hailey disease, porphyria or amyloidosis.

13. (canceled)

14. (canceled)

15. (canceled)

16. (canceled)

17. (canceled)

18. (canceled)

19. (canceled)

20. (canceled)

21. (canceled)

22. (canceled)

23. The method of claim 1, wherein the topical administration is in the form of a pharmaceutical composition and wherein said pharmaceutical composition is in the form of a gel, a cream, an ointment, an emulsion, an emu-gel, a foam, a suspension or a spray-patch.

24. (canceled)

25. The method of claim 1, wherein the topical administration comprises of 0.01-5 wt % of an alpha-2 adrenoreceptor agonist.

26. (canceled)

27. (canceled)

28. (canceled)

29. (canceled)

30. (canceled)

31. The method of claim 1, wherein the treatment comprises administration of an alpha-2 adrenoreceptor agonist as the sole active agent.

32. The method of claim 1, which further comprises the administration of at least one additional active agent.

33. The method of claim 32, wherein the additional agent is a corticosteroid, doxepine, tacrolimus, pimecrolimus, pramoxine, lidocaine, prilocaine, ketamine, amitriptyline, capsaicin, menthol, camphor, strontium, tofacitinib, crisaborole, N-palmitoylethanolamine, an antihistamine, an SNRI, an SSRI, naltrexone, butophanol, nalfurafine, gabapentin, pregabalin, aprepitant, thalidomide, lenalidomide, ursodeoxycholic acid, rifampin, cholestyramine, phenobarbital, Botulinum toxin A, naloxone, ASN008, SNA-125, TS-022, KPL-716 or orvepitant.

34. The method of claim 32, wherein the administration of the additional active agent is topical, oral, or parenteral.

35. (canceled)

36. The method of claim 1, wherein administration is once or twice daily.

37. (canceled)

38. A method of treating pruritus comprising the topical administration, to a subject in need thereof, of a therapeutically effective, substantially non-systemic amount of an alpha-2 adrenoreceptor agonist.

39. The method of claim 38, wherein the alpha-2 adrenoreceptor agonist is s apraclonidine, brimonidine, clonidine, detomidine, dexmedetomidine, guanabenz, lofexidine, medetomidine, romifidine or xylazine.
